(19) United States
(12) Patent Application Publication
(10) Pub. No.: US 2007/0292529 A1
(43) Pub. Date: Dec. 20, 2007

(54) Strontium compositions and methods of treating osteoporotic conditions

(76) Inventor: Philip S. Tabbiner, Wilmington, NC (US)

Correspondence Address: HUTCHISON LAW GROUP PLLC, PO BOX 31686, RALEIGH, NC, 27612, US

(21) Appl. No.: 11/455,605

(22) Filed: Jun. 19, 2006

(51) Int. Cl. A61K 33/42; A61K 33/06; A61K 31/185; A61K 31/19; A61K 31/714; A61K 31/525; A61K 31/51

(52) U.S. Cl. 424/601; 424/682; 514/52; 514/251; 514/276; 514/350; 514/356; 514/569; 514/574; 514/553; 424/472

(57) ABSTRACT

A therapeutic dosage form and method for treating an osteoporitic condition is described. The therapeutic dosage form comprises a divalent cationic source of strontium, vitamin B₆, vitamin B₁₂ and folic acid or folate. The method comprises administering to a subject in need thereof a therapeutic dosage form comprising a divalent cationic source of strontium, vitamin B₆, vitamin B₁₂ and folic acid or folate.

### FIELD OF THE INVENTION

This invention relates to therapeutic dosage forms for and therapeutic treatments of the symptoms and etiology of osteoporotic conditions.

### BACKGROUND

Bone consists of living cells widely scattered within a non-living material known as bone matrix. Two main types of cells are responsible for bone remodeling: the osteoblasts involved in bone formation and the osteoclasts involved in bone resorption. The matrix is formed by the action of osteoblasts—cells that make and secrete bone matrix proteins such as collagen, which provide elasticity, as well as mineral salts formed from calcium and phosphorous, which impart hardness to bone. As bone tissue matures, some osteoblasts are trapped in the bone matrix and differentiate into osteocytes, which are mature bone cells that carry out normal cellular activities. These osteocytes connect with other osteocytes through the bone matrix and can sense pressure or cracks in the bone. They therefore assist in directing where osteoclasts will act during the repair and/or regeneration of bone. Osteoclasts are cells that act to dissolve existing bone, thus facilitating bone growth, repair and regeneration.

Osteoporosis is a disease of worldwide concern in which bones become fragile and more likely to break. Osteoporosis develops when bone resorption occurs too rapidly, if bone replacement occurs too slowly, or due to a combination of both. This is in part due to the fact that it requires six months for osteoblasts to rebuild the amount of bone destroyed by osteoclasts in three days. In contrast, bone injury involves localized trauma to the bone. If not prevented or if left untreated, osteoporosis can progress painlessly until a bone breaks. These broken bones, also known as fractures, occur typically in the hip, spine, and wrist. Any bone can be affected, but of special concern are fractures of the hip and spine. A hip fracture almost always requires hospitalization and major surgery. It can impair a person's ability to walk unassisted and may cause prolonged or permanent disability or even death. Spinal or vertebral fractures also have serious consequences, including loss of height, severe back pain, and deformity along with the associated medical costs.

Osteoporosis is a major health and financial threat for 44 million Americans, 68% of whom are women. At least one half of adult women and 1 in 5 adult men over the age of 50 will sustain one or more vertebral, hip or other fractures. The annual social care and acute costs for treating osteoporotic related injuries is estimated to be over 14 billion dollars and is expected to increase. Osteoporosis is diagnosed by a bone mineral density (BMD) test, which is a qualitative way to detect low bone density. There is no cure for osteoporosis; however, several drugs and medication options are approved for the prevention and treatment of osteoporosis.

Arthritis is a condition that affects the joints and surrounding tissues. The two most common types of arthritis are osteoarthritis and rheumatoid arthritis. While it is possible to have both osteoporosis and an arthritic condition, people with osteoarthritis may be less likely to develop osteoporosis. On the other hand, people with rheumatoid arthritis may be more likely to develop osteoporosis, especially as a secondary condition from drugs used in rheumatoid arthritis treatment. Therefore there is a need to develop treatments and regimens for the treatment of these conditions individually and concurrently.

### SUMMARY

In one embodiment, a therapeutic dosage form is provided comprising a divalent cationic source of strontium of at least about 50 milligrams, vitamin B₆, vitamin B₁₂ and folic acid or folate.

In another embodiment, a method is provided for treating symptoms or etiology of osteoporosis in a subject. The method comprises the step of administering to a mammal in need thereof a therapeutically effective amount of a therapeutic dosage form comprising a divalent cationic source of strontium, vitamin B₆, vitamin B₁₂ and folic acid or folate.

Utilization of the combination of substances in conformance with the present method may facilitate an increase in bone mass and/or an increase in mobility of the joints, and/or a reduction of the levels of pain. The forgoing and other features, advantages and embodiments of the present invention may be more fully appreciated by reference to the following detailed description.

### DETAILED DESCRIPTION OF THE EMBODIMENTS

As used herein, “pharmacologically active agent” or “active agent” are used interchangeably and refer to a compound or composition of matter which, when administered to a human or animal induces a desired pharmacologic and/or physiologic effect by local and/or systemic action.

As used herein, “therapeutically effective amount” refers to an amount of an active agent that is nontoxic but sufficient to provide the desired effect. For example, a therapeutically effective amount of a divalent cationic source of strontium is an amount sufficient to measurably decrease the symptom or etiology of an osteoporitic condition. The therapeutically effective amount varies according to the patient's sex, age and weight, the route of administration, the nature of the condition and any treatments which may be associated therewith, or any concurrent related or unrelated treatments or conditions of the patient. Therapeutically effective amounts can be determined without undue experimentation by any person skilled in the art or by following the exemplary guidelines set forth in this application.

As used herein, “pharmaceutical dosage form” refers to a dosage form of an active agent (e.g., tablet, film, injectable, powder, capsule, and the like) which is generally safe, non-toxic and neither biologically nor otherwise undesirable. A pharmaceutical dosage form includes that which is acceptable for veterinary use as well as human pharmaceutical use, and which possesses the necessary and desirable characteristics of a dosage form acceptable for administration to a patient (e.g., a tablet of acceptable hardness, dissolution, stability, and a size and weight practical for oral administration). A pharmaceutical dosage form may include multiple tablets or capsules each of which comprises some part or fraction of the active agents for patient administration such that the multiple tablets or capsules taken together comprise the pharmaceutical dosage form.

The present embodiments relate to therapeutic dosage forms and tablets containing as active agents a divalent cationic source of strontium, vitamin B₆, vitamin B₁₂ and folic acid or folate. Further additional optional ingredients may be added as described above.

The therapeutic dosage forms may contain from about 250 milligrams to about 2000 milligrams of total active agents, or from about 500 milligrams to about 1500 milligrams of total active agents, or from about 500 milligrams to about 1250 milligrams of total active agents. The therapeutic dosage form may be administered orally, rectally or parenterally at a dose 250 to about 2000 milligrams of total active agents per day, or from about 500 milligrams to about 1500 milligrams of total active agents per day, or from about 500 milligrams to about 1250 milligrams of total active agents per day. The therapeutic dosage form may be administered in one or more therapeutic dosage forms, for example, one or more tablets or capsules per day.

The effective dosage amount may be determined by routine experimentation, for example, by performing various pharmacological studies with regard to anti-resorbent properties in mammalian models. Anti-resorbent bone properties may be determined on mice calvaria in the presence or absence of the active agents of the therapeutic dosage forms herein described according to a model based on the method described by Reynolds and Dingle, “A sensitive in vitro method for studying the induction and inhibition of bone resorption,” Calc. Tiss. Res., 4, 339-349 (1970).

The term “divalent cationic source of strontium” refers generally to salts of strontium and specifically to when strontium is a divalent cation, and is therefore independent of the nature of the anion. By way of example, about 650 milligrams of strontium gluconate is approximately 119 milligrams of a divalent cationic source of strontium (e.g., atomic weight of strontium÷formula weight of strontium salt)×(weight of strontium salt)=weight of divalent cationic source of strontium). Divalent cationic sources of strontium may include distrontium salts. Strontium and distrontium salts may include any and all hydrates thereof and mixtures of hydrates. By way of example, a source of divalent cationic strontium may be strontium acetyl salicylate, strontium acetyloxy-benzoate, strontium ascorbate, strontium aspartate in either L and/or D-form, strontium benzenesulfonate, strontium butyrate, strontium camphorate, strontium carbonate, strontium clodronate, strontium chloride, strontium citrate, strontium ethanesulfonate, strontium fumarate, strontium gluconate, strontium glutamate in either L- and/or D-form, strontium glutarate, strontium ibandronate, strontium ibuprofenate, strontium ketoprofenate, strontium lactate, strontium L-threonate, strontium malate, strontium maleate, strontium maleate, strontium malonate, strontium methanesulfonate, strontium naproxenate, strontium nitrate, strontium oxalate, strontium phosphate, strontium pyruvate, strontium ranelate, strontium risedronate, strontium salicylate, strontium succinate, strontium sulfate, strontium tartrate and mixtures thereof. The divalent cationic source of strontium may be at least 50 milligrams.

As used herein, vitamin B₆ refers to pyridoxine HCl, pyridoxal, pyridoxal HCl, pyridoxal 5-phosphate, pyridoxal 5-phosphate calcium salt, pyridoxamine, pyridoxamine dihydrochloride, pyridoxamine phosphate and mixtures thereof.

As used herein, “vitamin B₁₂” refers to cobalamins comprising a 5,6-dimethylbenzimidazole heterocyclic base and derivatives, analogs and coenzymatically active forms thereof. For example, vitamin B₁₂ includes cobalamin, cobamide, cyanocobalamin, aquacobalamin, hydroxocobalamin, co-methylcobalamin and nitritocobalamin and mixtures thereof.

As used herein, folic acid or folates are synonymous with pteroylglutamic acid and pteroylglutamate, respectively. The term folates refers to any member of the family of pteroylglutamates, (D or L isomers and racemates) or mixtures of them, having various levels of reduction of the pteridine ring, one-carbon substitutions, substitutions on the glutamate residues and mixtures thereof.

As used herein, tablet or capsules refers generally to solid, gelatinous dosage forms containing active agents with or without suitable diluents and prepared either by compression or molding methods known in the art. Tablets may be discoid in shape, or they may also be round, oval, oblong, cylindrical, or triangular. Tablets may include buccal forms, sublingual forms, oral disintegrating forms and oral care strips. They may differ in size and weight depending on the amount of active agents present and the intended method of administration. The tablets may be compressed tablets, molded tablets or tablet triturates. Tablets may include coated tablets, sugar-coated tablets, buccal tablets, oral disintegrating tablets, and sublingual tablets, or in other forms. Buccal drug delivery, e.g., delivery of a drug by passage of a drug through the buccal mucosa into the bloodstream, may be affected by placing a buccal dosage form on the upper gum or opposing inner lip area of the subject. For buccal administration, tablets or lozenges formulated in the conventional manner may be used. Penetration enhancers or permeation enhancers to an increase the permeability of the buccal mucosal tissue to a pharmacologically active agent, e.g., so that the rate at which the drug permeates through the mucosal tissue is increased, may be included therein.

Subjects who may have difficulty swallowing a large tablet, due to esophageal strictures or other pathology, for example, may be administered a therapeutically effective solution of the active agents herein disclosed via a suspension in a pharmaceutically acceptable carrier. Alternatively, such subjects may be provided a liquid, buccal or sublingual form or an oral care strip to be introduced to the oral mucosa.

In addition to the pharmacologically active agent, pharmaceutical dosage forms may contain a number of inert materials or additives. Inert materials and additives may include materials that help in the manufacture of the tablet or to impart satisfactory compression characteristics to the formulation. Inert materials and additives may also include materials that help to give additional desirable physical characteristics to the finished tablet, such as colors, flavors, and sweetening agents. Such inert materials and additives should not materially affect the pharmacological properties of the active agent or agents.

Pharmaceutical dosage forms, e.g., tablets or capsules, may contain one or more excipients or vehicles chosen from diluents, lubricants, binders, disintegrating agents, absorbents, and the like. By way of example and without limitation the diluents may include lactose, dextrose, sucrose, mannitol, sorbitol, cellulose, and/or glycerin. The lubricants may include silica, talc, stearic acid and its magnesium and calcium salts, and/or polyethyleneglycol. The binders may include aluminum and magnesium silicate, starch, gelatin, tragacanth, methyl-cellulose, sodium carboxymethylcellulose and/or polyvinylpyrrolidone.

The dissolution rate of a pharmaceutical dosage form, e.g., tablet or capsule, may be increased by the addition of disintegrant or solubilizing substances or effervescent mixtures, such as, for example, alginic acid, amylose, amylose croscarmellose sodium, calcium alginate, calcium carbonate, calcium phosphate, carboxymethylcellulose, carboxymethylcellulose calcium, crospovidone, formaldehyde gelatine, lowly-substituted hydroxypropylcellulose, magnesium peroxide, pectic acid, powdered agar-agar, sodium bicarbonate, sodium carbonate, sodium carboxymethyl starch or starch, and other functionally equivalent substances. The dissolution rate of a tablet or capsule may be also controlled by processing the contents into granulated forms, pellets, or other forms, by addition of binders, dissolution-control agents, or other excipients. The active substance may be contained in the pharmaceutical dosage form not only as a solid but also in solution or in suspension, e.g. in vegetable oil, polyethyleneglycol or glycerol, using surfactants, etc.

The pharmaceutical dosage form may include an enteric coating. As used herein, enteric coating refers to pharmaceutical controlled release methods to deliver a therapeutic dosage form to the gastrointestinal tract with a desired level of effective amount of active agents without the adverse gastrointestinal effects. Enteric coatings may be pH sensitive polymers designed to remain intact in the acidic environment of the stomach, but to dissolve in the more alkaline environment of the intestine. Enteric coatings may include by way of example, blends of cellulose acetate phthalate polymers (CAP), (see Wu et al, U.S. Pat. No. 5,356,634), cellulose acetate trimellitate polymers (CAT), (see Crook et al, U.S. Pat. No. 5,723,151), polyvinylpyrrolidone (PVP) with CAP and diethyl phthalate coating, (see Sipos, U.S. Pat. No. 4,079,125), polymers of an acrylic resin and an undercoat and overcoat of PVP, (see Patell, U.S. Pat. No. 4,775,536) and hydroxypropylmethyl cellulose phthalate (see Hodges et al, U.S. Pat. No. 5,225,202). Other enteric coatings may include hydroxypropyl methylcellulose phthalate (HPMCP), hydroxypropyl methylcellulose acetate succinate (HPMCAS), polyvinyl acetate phthalate (PVAP) and acrylic resins. Preferably, disintegration of the enteric coating occurs in approximately 40 minutes, to correspond with the approximate time the therapeutic dosage form enters or is in the intestine.

As used herein, the terms “acceptable carrier” or “pharmaceutically acceptable carrier” are used interchangeably and refer to tablets, capsules, solvents, dispersion mediums, coatings, enteric coatings or delivery vehicles which may be used to administer the therapeutic dosage forms described herein without undue adverse physiological effects.

In one embodiment, a therapeutic dosage form may be provided comprising a divalent cationic source of strontium present in the amount of at least about 50 milligrams, vitamin B₆, vitamin B₁₂ and folic acid or folate.

In another embodiment, a therapeutic dosage form may be provided comprising a divalent cationic source of strontium in the amount of from about 50 milligrams to about 400 milligrams, vitamin B₆ in the amount of from about 10 milligrams to about 50 milligrams, vitamin B₁₂ in the amount of from about 1 milligrams to about 3 milligrams and folic acid or folate in the amount of from about 0.5 milligrams to about 3 milligrams.

The exact mechanism of biological action which may account for increased bone mass or reduction of bone mass loss and/or alleviation of arthritic induced pain and inflammation when a divalent cationic source of strontium, vitamin B₆, vitamin B₁₂ and folic acid or folate is used is not known. However, this combination of active agents is believed to contribute to the prevention of bone mass loss, cellular regeneration, muscular tissue maintenance and tissue recovery acceleration and thus account for its potential beneficial effects in controlling and/or preventing osteoporosis, and may also contribute to effective treatment and relief of osteoarthritic conditions and arthritic inflammation when administered as herein disclosed. Increased bone mass or reduction of bone mass loss by the therapeutic dosage form described herein may include, for example, generating new or additional bone at locations where such bone growth is not presently taking place and/or stimulating the growth of bone which is already in the process of formation. Increased bone mass or reduction of bone mass loss may be the result of the combined effects of the divalent source of strontium and vitamin B₆, vitamin B₁₂ and folic acid or folate by increasing osteoblast activity in the subject and may further be coupled with an elevation at least one bone anabolic agent in the subject. An example of a bone anabolic agent endogenously produced in the human body may be parathyroid hormone (PTH). The effects of the divalent source of strontium and vitamin B₆, vitamin B₁₂ and folic acid or folate on increased bone mass or reduction of bone mass loss in combination with alleviating or eliminating possible inflammation may provide a more desirable and/or effective regimen. For example, for the divalent source of strontium and vitamin B₆, vitamin B₁₂ and folic acid or folate may be effective for treating subjects suffering from both osteoporosis and rheumatoid arthritis.

The divalent cationic source of strontium, vitamin B₆, vitamin B₁₂ and folic acid or folate may have advantages over strontium compositions alone. The divalent cationic source of strontium, vitamin B₆, vitamin B₁₂ and folic acid or folate may have advantages over vitamin B₆, vitamin B₁₂ and folic acid or folate compositions alone. For example, potentially improved bioavailability and simultaneous treatment of bone/joint aliments or degeneration may make it possible to administer reduced dosages of therapeutics in the treatment of osteoporosis and/or arthritic conditions, particularly when both osteoporosis and arthritic conditions are present in a subject.

In another embodiment, a tablet is provided comprising a therapeutically effective amount of a divalent cationic source of strontium, vitamin B₆, vitamin B₁₂ and folic acid or folate. In the tablet, the therapeutically effective amount of a divalent cationic source of strontium may be present in the amount of from about 50 milligrams to about 400 milligrams, vitamin B₆ may be present in the amount of from about 10 milligrams to about 50 milligrams, vitamin B₁₂ may be present in the amount of from about 1 milligrams to about 3 milligrams and folic acid or folate may be present in the amount of from about 0.5 milligrams to about 3 milligrams. In an exemplary embodiment, the tablet comprises a source of strontium in the amount of from about 50 milligrams to about 400 milligrams, vitamin B₆ in amount of about 25 milligrams, vitamin B₁₂ in the amount of about 2 milligrams, and folic acid or folate in the amount of about 1.5 milligrams.

Other optional vitamins and mineral components may be included in the therapeutic dosage form and method. These additional components may include iodine, calcium, potassium, iron, magnesium, manganese, zinc and selenium, preferably in the form of chelates, vitamins A, D, E, choline bitartrate, inositol, pantothenic acid, nicotinic acid, biotin, rutin, betain, α-lipoic acid and glutamic acid.

In addition to a divalent cationic source of strontium, vitamin B₆, vitamin B₁₂ and folic acid or folate, the therapeutic dosage form may further include one or more therapeutically and/or prophylactically active substances. Such active substances may include agents effective in the treatment of or acting on joint tissue components or bone, or for increasing bone mass or reducing bone mass loss. For example, and without limitation, such agents may include anabolic agents, analgesic agents, antiresorptive agents aromatase inhibitors, chondroitin sulphate, COX-2 inhibitors, COX-3 inhibitors, disease modifying anti-rheumatic compounds (DMARDs), glucocorticoids, glucosamine, glycine antagonists, inhibitors of inducible nitric oxide synthetase (iNOS), inhibitors of interleukin-1 converting enzyme, inhibitors of matrix metallo-proteinases (MMPs), inhibitors/antagonists of IL-1, inhibitors/antagonists of RANK-ligand, inhibitors/antagonists of TNF-oc, N-acetylcholine receptor agonists, neurokinin antagonists, neuroleptic agents, NMDA receptor antagonists, non-steroidal anti-inflammatory agents (NSAIDs), opioids, pallitative agents, PAR2 receptor antagonists, selective estrogen receptor modulators (SERMs) and vanilloid receptor antagonists. Additional therapeutically and/or prophylactically active substances may include adjuvants, alpha-lipoic acid, anti-infective agents, anti-inflammatory agents, antioxidants, glycosaminoglycans, herbal derivatives, vitamin E and mixtures thereof. Anabolic agents may include natural and truncated forms of parathyroid hormone (PTH) including aminated natural and truncated forms thereof, anabolic Vitamin D analogs, a low-density lipoprotein receptor-related protein 5, an activator of non-genomic estrogen-like signaling, a bone morphogenic protein (BMP), an insulin-like growth factor (IGF), a fibroblast growth factor (FGF), sclerostin, leptin, a prostaglandin, a statin, a growth hormone, a growth hormone releasing factor (GHRF), hepatocyte growth factor (HGF), calcitonin gene related peptide (CGRP), parathyroid hormone related peptide (PTHrP), transforming growth factor (TGF)-β1 and combinations thereof. Antiresorptive agents may include human calcitonin, non-human calcitonin, calcitonin gene related peptide (CGRP), hormone replacement therapy (HRT) agents such as selective estrogen receptor modulators, bisphosphonates, cathepsin-K inhibitors, and various combinations thereof.

In one embodiment, a method is provided for treating symptoms or etiology of osteoporosis in a subject. The method comprises the step of administering to a mammal in need thereof a therapeutically effective amount of a therapeutic dosage form comprising a divalent cationic source of strontium, vitamin B₆, vitamin B₁₂ and folic acid or folate.

The method may comprise the step of administering a therapeutic dosage form comprising a divalent cationic source of strontium in the amount of from about 50 milligrams to about 400 milligrams, vitamin B₆ is in the amount of from about 10 milligrams to about 50 milligrams, vitamin B₁₂ is in the amount of from about 1 milligrams to about 3 milligrams and folic acid or folate is in the amount of from about 0.5 milligrams to about 3 milligrams. In an exemplary embodiment of the method, the divalent source of strontium is in the amount of from about 100 milligrams to about 400 milligrams, vitamin B₆ is in the amount of about 25 milligrams, vitamin B₁₂ is in the amount of about 2 milligrams, and folic acid or folate is in the amount of about 1.5 milligrams

In one embodiment, the method may comprise orally administering to a subject in need thereof, a divalent cationic source of strontium in the amount of about 120 mg, vitamin B₆ in the amount of about 25 mg, B₁₂ in the amount of about 2 milligrams and folic acid in the amount of about 1.5 milligrams as a one tablet daily regimen. The method may comprise the step of administering orally a daily regimen of a single tablet comprising a divalent cationic source of strontium in the amount of about 120 mg, vitamin B₆ in the amount of about 25 mg, B₁₂ in the amount of about 2 mg, and folic acid in the amount of about 1.5 mg.

Utilization of a divalent cationic source of strontium, vitamin B₆, vitamin B₁₂ and folic acid or folate described hereinabove, may result in increase of bone mass, and/or increase of joint mobility and/or reduction of pain for most individuals in need thereof.

In addition to strontium compounds, vitamin B₆, vitamin B₁₂ and folic acid or folate, the method of treating symptoms or etiology of osteoporosis in a subject in need thereof may further include in the therapeutic dosage form one or more therapeutically and/or prophylactically active substances. Such active substances may include agents effective in the treatment of or acting on joint tissue components or bone or for increasing bone mass or reducing bone mass loss. For example, and without limitation, such agents may include anabolic agents, analgesic agents, antiresorptive agents aromatase inhibitors, chondroitin sulphate, COX-2 inhibitors, COX-3 inhibitors, disease modifying anti-rheumatic compounds (DMARDs), glucocorticoids, glucosamine, glycine antagonists, inhibitors of inducible nitric oxide synthetase (iNOS), inhibitors of interleukin-1 converting enzyme, inhibitors of matrix metallo-proteinases (MMPs), inhibitors/antagonists of IL-1, inhibitors/antagonists of RANK-ligand, inhibitors/antagonists of TNF-oc, N-acetylcholine receptor agonists, neurokinin antagonists, neuroleptic agents, NMDA receptor antagonists, non-steroidal anti-inflammatory agents (NSAIDs), opioids, pallitative agents, PAR2 receptor antagonists, selective estrogen receptor modulators (SERMs) and vanilloid receptor antagonists. Additional therapeutically and/or prophylactically active substances may include adjuvants, alpha-lipoic acid, anti-infective agents, anti-inflammatory agents, antioxidants, glycosaminoglycans, herbal derivatives, vitamin E and mixtures thereof. Anabolic agents may include natural and truncated forms of parathyroid hormone (PTH) including aminated natural and truncated forms thereof, anabolic Vitamin D analogs, a low-density lipoprotein receptor-related protein 5, an activator of non-genomic estrogen-like signaling, a bone morphogenic protein (BMP), an insulin-like growth factor (IGF), a fibroblast growth factor (FGF), sclerostin, leptin, a prostaglandin, a statin, a growth hormone, a growth hormone releasing factor (GHRF), hepatocyte growth factor (HGF), calcitonin gene related peptide (CGRP), parathyroid hormone related peptide (PTHrP), transforming growth factor (TGF)-β1 and combinations thereof. Antiresorptive agents may include human calcitonin, non-human calcitonin, calcitonin gene related peptide (CGRP), hormone replacement therapy (HRT) agents such as selective estrogen receptor modulators, bisphosphonates, cathepsin-K inhibitors, and various combinations thereof.

### EXAMPLES

The following examples are illustrative of the embodiments of the present invention and are not to be interpreted as limiting or restrictive. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements (e.g., weights). Thus, in one example, a therapeutic dosage form may be prepared by combining the following: about 650 milligrams of strontium gluconate (approximately 120 milligrams of divalent cationic strontium); about 25 milligrams of vitamin B₆; about 2 milligrams of vitamin B₁₂; and about 1.5 milligrams of folic acid along with acceptable excipients; and processed into tablets. The tablets may further include enteric coatings. A daily regimen of one tablet as just described may provide effective treatment to a subject with an osteoporitic condition or a subject with an osteoporitic condition and an arthritic condition, such as rheumatoid arthritis.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

While the invention has been described in detail and with reference to specific embodiments thereof, it will be apparent to one skilled in the art that various changes and modifications can be made without departing from the spirit and scope of the invention.

What is claimed is:

1. A therapeutic dosage form comprising a divalent cationic source of strontium of at least about 50 milligrams, vitamin B₆, vitamin B₁₂ and folic acid or folate.

2. The therapeutic dosage form of claim 1, wherein the divalent cationic source of strontium is in the amount of from about 50 milligrams to about 400 milligrams, vitamin B₆ is in the amount of from about 10 milligrams to about 50 milligrams, vitamin B₁₂ is in the amount of from about 1 milligrams to about 3 milligrams and folic acid or folate is in the amount of from about 0.5 milligrams to about 3 milligrams.

3. The therapeutic dosage form of claim 1, wherein the source of divalent strontium is selected from the group consisting of: strontium acetyl salicylate, strontium acetyloxy-benzoate, strontium ascorbate, strontium aspartate in either L and/or D-form, strontium benzenesulfonate, strontium butyrate, strontium camphorate, strontium carbonate, strontium clodronate, strontium chloride, strontium citrate, strontium ethanesulfonate, strontium fumarate, strontium gluconate, strontium glutamate in either L- and/or D-form, strontium glutarate, strontium ibandronate, strontium ibuprofenate, strontium ketoprofenate, strontium lactate, strontium L-threonate, strontium malate, strontium maleate, strontium maleate, strontium malonate, strontium methanesulfonate, strontium naproxenate, strontium nitrate, strontium oxalate, strontium phosphate, strontium pyruvate, strontium ranelate, strontium risedronate, strontium salicylate, strontium succinate, strontium sulfate, strontium tartrate and mixtures thereof.

4. The therapeutic dosage form of claim 1, wherein the divalent source of strontium is in the amount of from about 50 milligrams to about 400 milligrams, vitamin B₆ is in the amount of about 25 milligrams, vitamin B₁₂ is in the amount of about 2 milligrams and folic acid or folate is in the amount of from about 1.5 milligrams.

5. The therapeutic dosage form of claim 1, wherein the therapeutically effective therapeutic dosage form further comprises an acceptable carrier suitable for oral administration.

6. The therapeutic dosage form of claim 1, wherein the therapeutically effective therapeutic dosage form further comprises an enteric coating such that the therapeutically effective therapeutic dosage form is controllably released into the gastrointestinal tract when administered orally.

7. A method for treating symptoms or etiology of osteoporosis in a subject comprising the step of administering to a mammal in need thereof a therapeutically effective amount of a therapeutic dosage form comprising a divalent cationic source of strontium, vitamin B₆, vitamin B₁₂ and folic acid or folate.

8. The method of claim 7, wherein the a divalent cationic source of strontium is in the amount of from about 50 milligrams to about 400 milligrams, vitamin B₆ is in the amount of from about 10 milligrams to about 50 milligrams, vitamin B₁₂ is in the amount of from about 1 milligrams to about 3 milligrams and folic acid or folate is in the amount of from about 0.5 milligrams to about 3 milligrams.

9. The method of claim 7, wherein the divalent source of strontium is in an amount of from about 100 milligrams to about 400 milligrams, vitamin B₆ is in amount of about 25 milligrams, vitamin B₁₂ is in the amount of about 2 milligrams, and folic acid or folate is in the amount of about 1.5 milligrams.

10. The method of claim 7, wherein the therapeutically effective amount of the divalent source of strontium is selected from the group consisting of: strontium acetyl salicylate, strontium acetyloxy-benzoate, strontium ascorbate, strontium aspartate in either L and/or D-form, strontium benzenesulfonate, strontium butyrate, strontium camphorate, strontium carbonate, strontium clodronate, strontium chloride, strontium citrate, strontium ethanesulfonate, strontium fumarate, strontium gluconate, strontium glutamate in either L- and/or D-form, strontium glutarate, strontium ibandronate, strontium ibuprofenate, strontium ketoprofenate, strontium lactate, strontium L-threonate, strontium malate, strontium maleate, strontium maleate, strontium malonate, strontium methanesulfonate, strontium naproxenate, strontium nitrate, strontium oxalate, strontium phosphate, strontium pyruvate, strontium ranelate, strontium risedronate, strontium salicylate, strontium succinate, strontium sulfate, strontium tartrate and mixtures thereof.

11. The method of claim 7, further comprising administering a therapeutically effective amount of one or more members selected from the group consisting of anabolic agents, analgesic agents, antiresorptive agents aromatase inhibitors, chondroitin sulphate, COX-2 inhibitors, COX-3 inhibitors, disease modifying anti-rheumatic compounds (DMARDs), glucocorticoids, glucosamine, glycine antagonists, inhibitors of inducible nitric oxide synthetase (iNOS), inhibitors of interleukin-1 converting enzyme, inhibitors of matrix metallo-proteinases (MMPs), inhibitors/antagonists of IL-1, inhibitors/antagonists of RANK-ligand, inhibitors/antagonists of TNF-oc, N-acetylcholine receptor agonists, neurokinin antagonists, neuroleptic agents, NMDA receptor antagonists, non-steroidal anti-inflammatory agents (NSAIDs), opioids, pallitative agents, PAR2 receptor antagonists, selective estrogen receptor modulators (SERMs), vanilloid receptor antagonists, adjuvants, alpha-lipoic acid, anti-infective agents, anti-inflammatory agents, antioxidants, glycosaminoglycans, herbal derivatives, natural and truncated forms of parathyroid hormone (PTH), aminated natural and truncated forms of parathyroid hormone (PTH), anabolic Vitamin D analogs, low-density lipoprotein receptor-related protein 5, non-genomic estrogen-like signaling activator, bone morphogenic protein (BMP), insulin-like growth factor (IGF), fibroblast growth factor (FGF), sclerostin, leptin, a prostaglandin, statin, growth hormone, growth hormone releasing factor (GHRF), hepatocyte growth factor (HGF), calcitonin gene related peptide (CGRP), parathyroid hormone related peptide (PTHrP), transforming growth factor (TGF)-β1, human calcitonin, non-human calcitonin, calcitonin gene related peptide (CGRP), hormone replacement therapy (HRT) agents, selective estrogen receptor modulator, bisphosphonates, and cathepsin-K inhibitors.

12. The method of claim 7, wherein the therapeutically effective therapeutic dosage form further comprises an acceptable carrier suitable for oral administration.

13. The method of claim 7, wherein the therapeutically effective therapeutic dosage form further comprises an enteric coating such that the therapeutically effective therapeutic dosage form is controllably released into the gastrointestinal tract when administered orally.
